(19) United States
(12) Patent Application Publication
(10) Pub. No.: US 2012/0177603 A1
(43) Pub. Date: Jul. 12, 2012

(54) METHOD FOR THE PURIFICATION OF INTERFERON-B

(75) Inventors: Stefan Arnold, Schwetzingen (DE); Christian Scheckermann, Ehrenkirchen/Ehrenstatten (DE)

(73) Assignee: BioGenerix AG, Mannheim (DE)

(21) Appl. No.: 13/382,807

(22) PCT Filed: Jul. 7, 2010
(86) PCT No.: PCT/EP10/04130

§ 371 Date: Mar. 28, 2012

(30) Foreign Application Priority Data
Jul. 7, 2009 (DE) 10200932179.9

(51) Int. Cl. A61K 38/21; C07K 14/565

(52) U.S. Cl. 424/85.6; 530/351

(57) ABSTRACT

A method for the production of human glycosylated Interferon-beta (IFN-β) is described, comprising two affinity chromatography steps followed by hydrophobic interaction chromatography step preferably with a subsequent anion exchange chromatography step. IFN-β obtained by the method as described is characterized by a high purity and specific biologically activity, because of which it is particularly suitable for the production of pharmaceutical compositions.

The present invention relates to a method for the production of recombinant human Interferon-β (IFN-β), comprising at least one affinity chromatography (AC) and at least one hydrophobic interaction chromatography (HIC) step. In particular, the present invention relates to a method for the purification of glycosylated IFN-β from cell culture supernatant or a mixture of other proteins, comprising two affinity chromatography steps with subsequent hydrophobic interaction chromatography steps as preferably followed by an anion exchange chromatography (AEX) step.

### BACKGROUND OF THE INVENTION

Naturally occurring Interferons are species specific proteins, partially glycoproteins, which are secreted by different cell types of the body upon induction by viruses, double-stranded RNA, other polynucleotides as well as antigens. Interferons possess numerous biological activities such as antiviral, antiproliferative as well as immunomodulating properties. So far, at least three different types of human interferons have been identified, which are produced by leucocytes, lymphocytes, fibroblasts as well as cells of the immune system and designated α-, β-, and γ-interferons. Several interferon types are further subdivided in subtypes. Native human IFN-β can be industrially produced by superinduction of human fibroblast cell cultures with Poly-IC and subsequent isolation and purification of IFN-β by chromatographic and electrophoretic techniques. Applying recombinant DNA technology, proteins or polypeptides having comparable features as naturally occurring IFN-β can be produced; see for example European patent applications EP 028 033, EP 0 041 313, EP 070 906 and EP 287 075 as well as Chemajovsky et al. DNA 3 (1984), 297-308 and McCormick et al. Mol. Cell. Biol. 4 (1984), 166-172. In this way, human recombinant IFN-β can be produced by eukaryotic cells (for example CHO cells) or by prokaryotic cells (for example E. coli). The corresponding interferons are designated IFN-β-1a and IFN-β-1b, respectively. In contrast to IFN-β-1b, IFN-β-1a is glycosylated; see Goodkin, Lancet 344 (1994), 1057-1060.

A prerequisite for the therapeutic application of Interferon-β implies that it can be provided in sufficient amounts and high purity and it is formulated in a galenic composition, which makes the protein suitable for long-term storage by maintaining the molecular integrity. Interferon-β is instable and is subject to different degradation reactions. These include especially the cleavage of peptide bonds, deamidation, oxidation of methionine to methioninsulfide, disulfide exchange as well as modification of the sugar side chain up to deglycosylation.

Murine Interferon-β (IFN-β) differs significantly from human IFN-β. Therefore, the principles for purifying murine IFN-β described in the numerous available literature are not transferable and in the last decade, intensive efforts have been made to provide and optimize purification protocols for IFN-β, in order to isolate IFN-β in a sufficient amount and in a form in which it is suitable for stable storage and therapeutic use. Therefore, several publications exist, which describe different purification methods of IFN-β.

European patent application EP 011 435 discloses a sequence of purification steps comprising a cation exchange and a metal chelate affinity chromatography step.

European patent application EP 027 262 describes a method for purification comprising a dye ligand chromatography with Cibacron Blue.

European patent application EP 041 313 describes the use of a zinc chelate chromatography for purification of IFN-β.

European patent application EP 094 672 and EP 118 808 describe a purification method comprising Cibacron Blue and metal chelate chromatography.

European patent application EP 215 658 describes a sequence of purification steps comprising an affinity chromatography and a High Performance Liquid Chromatography (HPLC).

In European patent application EP 274 900 the purification of IFN-β inter alia by affinity chromatography and reverse phase HPLC (RP-HPLC) is described.

European patent application EP 467 992 describes the use of a metal chelate chromatography for the purification of IFN-β.

European patent application EP 529 300 discloses a sequence of purification steps comprising liquid/liquid phase extraction, Cibacron Blue affinity chromatography, immobilized Metal Ion Affinity Chromatography (IMAC) and gel chromatography.

International patent application WO98/28007 describes a sequence of purification steps comprising affinity chromatography, cation exchange chromatography and metal chelate chromatography.

German patent application DE 30 28 919 describes a sequence of purification steps comprising affinity chromatography and RP-HPLC.

In German patent application DE 30 39 566 the purification of IFN-β inter alia by glass absorption and a metal chelate chromatography step is described.

European patent application EP 446 850 describes a sequence of purification steps comprising glass absorption and cation exchange chromatography (CEX).

The purification of IFN-β by immunoaffinity chromatography is also known in the art; see for example Conradt et al. J. Biol. Chem. 262 (1987), 14600-14605, wherein a preceding anion exchange chromatography is performed.

Furthermore, the purification of IFN-β by lectin affinity chromatography, in particular by use of Concanavalin A (ConA), and a hydrophobic interaction chromatography with Phenyl-Sepharose has been described; see for example Carter and Horoszewicz, Pharmacol. Ther. 8 (1980), 359-377 as well as Mikulski et al. Prep. Biochem. 10 (1980), 103-119.

The incorporation of a Size Exclusion Chromatography (SEC) with a preceding dye ligand and metal chelate affinity chromatography step is described in the thesis of Meyer (2000), Faculty Chemistry of the University of Hannover.

Recently, for purification of interferons in general it has been suggested to use a cation exchange chromatography employing a solid matrix, comprising a more basic pH than the pH which corresponds to the isoelectric point (pI) of the interferons to be purified, in that at that pH the proteins are still absorbed and elution of said proteins takes place by increasing the ionic strength and/or the pH of the aqueous buffer solutions, see European patent application EP 1 273 592.

The object of the present invention is to provide a method for purifying biologically active recombinant human IFN-β in satisfactory purity and amount. Furthermore, the method should be simple and straightforward in realization. Desirable is a purification process which is applicable in routine process under GMP (Good Manufacturing Practice) aspects, and which preferably takes requirements for regulatory acceptance (validation, reproducibility) and particular biochemical peculiarities (such hydrophobicity) of IFN-β into account.

### DISCLOSURE OF THE PRESENT INVENTION

These and further technical problems are being solved by the method as characterized in claim 1 and as disclosed in the description and accompanying illustrated methods of the Examples. Preferred embodiments are described in the depended claims and further below.

It was found that by applying chromatographic purification of human recombinant IFN-β, yielded an acceptable purity and satisfactory yield of recombinant biologically active IFN-β by affinity chromatography and a hydrophobic interaction chromatography step and preferably an anion exchange chromatography step. The purity grade can be further increased by applying filtration steps.

Therefore, the present invention relates to a method for purification of recombinantly produced biologically active human IFN-β (IFN-β), comprising at least one affinity chromatography (AC) step and at least one hydrophobic interaction chromatography (HIC) step, wherein these chromatography steps can be performed immediately after another in either order.

Preferably, cell culture supernatant or cell fractions containing IFN-β serve as starting material for the chromatographic purification in order to reach a sufficient purity which allows for its application for formulation of a pharmaceutical composition. IFN-β intended for said purification is a polypeptide which exhibits biological and/or immunological features of naturally occurring human IFN-β and can be either a natural or a recombinant IFN-β. Preferably, glycosylated IFN-β, more preferably recombinant IFN-β from eukaryotic host cells, preferably CHO cells, is used. Most preferably, IFN-β species originated from the cell line BIC 8622 (ECACC 87 04 03 01) are used, which are for example described in European patent applications EP 287 075 and EP 529 300, the disclosures of which are referenced hereby.

IFN-β (previously: fibroblast-interferon) is a sialo-glycoprotein comprised of 166 amino acids, Mr=22-22.5 kD (polypeptide=18.5 kD), one N-glycosylation site at position 80 (Asn₈₀) with an average of two sialic acids/Mol (95% biantennary), three cysteins with one intramolecular disulfide bridge (Cys₃₁-Cys₁₄₁) and one free cystein residue (C₁₇). The correct formation of the disulfide bond is essential for the biological activity. The protein consists of 40% hydrophobic amino acids and is extremely hydrophobic (insoluble). Its pI is slightly basic (7.8-8.9). The amino acid sequence shows four histidine residues in position 93, 97, 121 and 131, which explains the good binding to Me⁺⁺ ligands.

The specific activity of IFN-β should be at least 2×10⁸ IU/mg. IFN-β preparations with high triantennary (>25%) and additional tetraantennary glycosylation (>5%) have been described, which can have a specific activity up to 3×10⁸ IU/mg and more. The two essential biological activities of IFN-β which can be measured are its antiviral and antiproliferative effect. Each of these biological activities can be measured by standard methods through inhibition of the cytopathic effect of a virus. A detailed description of the test methods used can be found in Stewart, W. E. 11 (1981), The Interferon System (Second, enlarged Edition), Springer-Verlag: Wien, New York; Grossberg, S. E. et al. (1984), Assay of Interferons. In: Came, P. E., Carter W. A (eds) Interferons and their Applications, Springer-Verlag: Berlin, Heidelberg, New York, Tokyo, pp. 23-43.

In a preferred embodiment of the present invention, the method of purifying IFN-β comprises two affinity chromatography steps, preferably performed before the hydrophobic interaction chromatography step. Experiments performed within the scope of the present invention surprisingly revealed that purification of IFN-β from cell culture supernatant through dye ligand affinity chromatography (AC), metal chelate affinity chromatography (MAC) and a hydrophobic interaction chromatography (HIC), as described hereinafter and in particular in the Examples was found to lead to an IFN-β preparation already substantially pure and stable in liquid formulation as well as in frozen or thawed state, and to have a high biological activity, comparable to commercial products like Avonex© (Biogen Idec) and Rebif© (Serono) or even better. According to RP-HPLC measurements, the metal chelate affinity chromatography (Zinc Sepharose chromatography) yields are 90-100%, and the hydrophobic interaction chromatography (Butyl Sepharose chromatography) yields are >70%, respectively. Analysis by RP-HPLC as well as SDS-PAGE and subsequent silver staining confirmed that IFN-β was purified up to apparent homogeneity. A band shift in SDS-PAGE under reduced and non-reduced conditions revealed that the internal disulfide bond was formed and thus the protein was correctly folded. In Isoelectric Focussing (IEF) Western blots the intermediate purification product and the final product exhibit a similar IFN-β isoform pattern as Avonex©.

The IFN-β preparation according to the present invention has been further analyzed via analytical SEC using Superdex 75 HR 10/30. Major elution peaks at A280 and A214 exhibit a peak maximum between 13.9 and 14.0 ml elution volume. The apparent molecular mass is 14 kDa, which indicates that the IFN-β monomers were eluted with a slight delay, probably due to an unspecific interaction with the column matrix.

At this stage the specific activity of the IFN-β purified according to the present invention already usually exhibits not less than 1×10⁸ IU/mg, typically exceeding at least 2×10⁸ IU/mg, and preferably exceeding at least 3×10⁸ IU/mg and more.

For removal of possibly existing remaining host cell DNA and potential viral contamination and other negatively charged contamination, in one further embodiment of the present invention the purification method for IFN-β comprises an anion exchange chromatography (AEX) step, preferably directly applied after the HIC step in flow through modus. This further AEX step is especially advantageous for the preparation of pharmaceuticals composition of IFN-β, since in control experiments with additional spiking of samples with virus material, the obtained IFN-β preparation was no longer infectious and therefore is suitable for therapeutic application.

By application of the above described chromatography steps, purified recombinant human IFN-β can be provided in a sufficiently pure grade and therefore, no additional chromatography step is necessary for the purification process of the present invention. Therefore, in a further embodiment of the purification method for IFN-β, a cation exchange chromatography (CEX) step is omitted. As mentioned in the description of the background to the present invention, particularly the use of a cation exchange chromatography for purification of interferon has been previously described. At first glance this step might be preferred to an anion exchange chromatography (AEX) step because of the relatively high pI (7.8-8.9) of IFN-β. However, since none or only weak binding takes place at a neutral pH (=pH in cell culture supernatant) and an acidification of the cell culture supernatant might lead to precipitation of other proteins necessitating additional filtration the CEX step is to be used as intermediary step and might be more suitable as a polishing step. However, CEX cannot be directly applied after an affinity chromatography step with dye ligands such as Cibacron or metal chelate as IMAC because it requires increasing salt concentrations for elution. Therefore, subsequent rebuffering and desalting steps would become necessary. Furthermore, in experiments performed in according with the present invention it turned out that the use of CEX technology yield only 25% of the total IFN-β amount present in the starting sample. For this reason, it is advantageous that the present invention does not make use of a CEX.

In a further preferred embodiment of the present invention, the method of purifying IFN-β does not comprises the use of a preparative HPLC. The same applies for reverse phase (RP) low or medium pressure chromatography which differs from the hydrophobic interaction chromatography according to the present invention, and which is preferably also not performed in the preparation of IFN-β in accordance with the method of the present invention. The omittance of this step in the preferred embodiment is advantageous since a HPLC step in the purification process is always associated with considerable effort. This is especially due to the expensive equipment (high investment) as well as required facilities because of additional constraints concerning personal security, flammable solvents, explosion protection etc. Therefore, RP-HPLC would be applied, if required, only for analytical purpose.

In a further embodiment, immunoglobulin affinity chromatography is not part of said purification process of the present invention. Those purification steps for therapeutic proteins are always associated with an extensive validation program in order to exclude security issues such as cross contamination. Therefore, omittance of said immunoaffinity steps is regarded as a big advantage.

In a further embodiment of the purification process of the present invention, the use of a hydroxyapatite chromatography is omitted.

Therefore, in a preferred embodiment the purification method according to the present invention makes only use of two to three different chromatography separation methods, in particular an affinity chromatography with dye ligands and/or metal chelates and a hydrophobic interaction chromatography, characterized by adsorption of the nonpolar surface regions of a protein at high salt concentrations to weak hydrophobic ligands in the stationary phase (salting effect) and elution by decreasing the buffer salt concentration. Optionally, but preferably this step is followed by an ion exchange chromatography step, based on the principle of a competitive interaction of charged ions, i.e. here anions.

This is to be distinguished from the chromatographic separation principle of a hydroxyapatite chromatography which is based on the use of anorganic hydroxyapatite crystals and therefore differs from the ion exchange chromatography such as an anion exchange chromatography and from the hydrophobic interaction chromatography. These chromatography principles are also clearly distinguished in the state of art; see for example Bioanalytik, F. Lottspeich, H. Zorbas (Editors), Heidelberg, Berlin, Spektrum Akad. Verlag 1998.

In a preferred embodiment, the chromatographic purification of IFN-β comprises the following steps:

(a) Dye affinity chromatography (AC) step;
(b) Metal chelate affinity chromatography (MAC) step;
(c) Hydrophobic interaction chromatography (HIC) step; and/or
(d) Anion exchange membrane filtration.

In this context, during the entire purification process the IFN-β sample should be kept in a cationic environment; i.e., at pH values below its isoelectric point (pI), so that the used buffers and washing solutions, apart from some individual washing steps, preferably exhibit a pH of ≦7; see also the Examples.

For the use of the dye affinity chromatography, for example Blue Dextran Sepharose R, or other suitable Cibacron R Blue immobilized matrices such as Matrex Gel Blue A from Amicon or Fraktogel 45 TSK AF-Blue from Merck or Blue-Sepharose R 6FF from GE Healthcare can be used.

Different matrices with chemical identical or different ligands can be used for the metal chelate chromatography. For the coordinative binding of recombinant IFN-β, suitable metal ions can be Cu²⁺, Zn²⁺, Co²⁺ or Ni²⁺ ions. The desorption can be induced by competitive substances such as imidazol, histidine, glycine or NH₄Cl, chelate agents as EDTA, IDA (iminodiacidic acid), TED (Tris-Carboxymethyl Ethylendiamine) or by lowering the pH value to pH 2 to 4.

Suitable separation media are immobilized iminodiacetic acid linked to agarose or to Fraktogel TSK HW-65F (Pierce) or Chelating Sepharose R FF (GE Healthcare) or Cellufine Chelate (Amicon).

As described above, the experiments performed according to the present invention revealed that particularly dye affinity chromatography with Cibacron Blue and IMAC chromatography, especially in combination, are particularly advantageous for the IFN-β purification process. Accordingly, in one preferred embodiment of the method of the present invention Cibacron Blue for the dye affinity chromatography (AC) step and Zn²⁺ chelate Sepharose (IMAC) for the metal chelate affinity chromatography (MAC) step is used.

The purification of IFN-β by means of a dye affinity chromatography with Cibacron Blue is already known in the art and has been frequently described; see the above described references, the disclosure content of which concerning the for performance of chromatography methods is enclosed herein by reference.

IFN-β is a strong binding partner of Cibacron Blue F3GA (CB-F3GA) and foreign bound proteins can be washed out by various buffers prior to elution. This strong interaction is most likely due to its enormous hydrophobicity. Since IFN-β quantitatively binds even at low concentrations under physiological conditions, this step is especially suitable as a capture step, i.e., as a first chromatography step of the purification process in accordance with the present invention. Elution of IFN-β can be conducted for example with ethyleneglycol, if necessary in a gradient. Blue Sepharose Streamliner or Blue Sepharose Fast Flow (GE Healthcare) are preferred materials.

Instead of or additionally to a dye affinity chromatography step also a lectin affinity chromatography step can be considered, for example using Concanavalin A (ConA). In experiments according to the present invention, it turned however out that ConA was difficult in handling and that no continuous substantial quality of the purified IFN-β could be provided because of qualitative differences of individual ConA batches. Therefore, it is preferred to omit lectin affinity chromatography step from the method of the present invention.

The immobilized metal chelate chromatography (IMAC) is also often described for purification of IFN-β. The carrier should be coupled with iminodiacetate (IDA). So far, IMAC was always performed after an affinity chromatography step (usually Cibacron Blue or ConA). Due to its high resolution this chromatography step is more suitable as an intermediate purification step. The strong binding can be explained by the presence of adjacent histidines in the amino acid sequence. IFN-β can be eluted using increasing salt combined with decreasing pH gradients. Salt on its own is not sufficient, however, IFN-β elutes at pH<5. For the above mentioned reason, use of gradients is recommended. In experiments performed in accordance with the present invention, it has been shown that especially Zn²⁺-charged chelating Sepharose Fast Flow (GE Healthcare) is well suited for the chromatography step in the purification process for IFN-β according to the present invention. As already explained herein, in a preferred embodiment of the purification process for IFN-β according to the present invention, a hydrophobic interaction chromatography (HIC) is performed, wherein preferably butyl groups serving as ligands. So far, the use of hydrophobic interaction chromatography for the purification of IFN-β has not been investigated in detail. Due to the extreme hydrophobicity of IFN-β, adsorption to and desorption from the hydrophobic matrix seemed to be problematical. In experiments conducted in accordance with the present invention, it was surprisingly found that IFN-β bound and easily eluted in the HIC step, especially when an acetate buffer with pH 5.0 is used for application and elution steps. As a further advantage of the method of the present invention, it turned out that HIC can be used as a capture step (requires addition of salt prior to applying the sample) and an intermediate chromatography step after the metal chelate chromatography such as IMAC and, if appropriate, can be performed directly after the dye affinity chromatography, e.g., with Cibacron. Furthermore, early inactivation of potentially present enzymes or viruses can be achieved in the HIC by elution with organic solvents.

There is a wide range of possible materials which can be used in HIC. In principle preferred for strongly hydrophobic proteins are short-chained alkyls like methyl, butyl or propyl, for example Butyl Sepharose 4 Fast Flow, Macro Prep Methyl, Fractogel EMD Propyl or Phenyl Sepharose Low Substitution (Merck). Since also the matrix contributes to binding, it had to be tested in the experiments according to the present invention, which material is finally best suited for the purification of IFN-β. In this context it turned out that butyl groups are most appropriate for absorption and in particular subsequent desorption; see also the Examples. Products of Amersham Biosciences (now GE Healthcare) can be used. The person skilled in the art can obtain product information on suitable matrices and protocols for performing hydrophobic interaction chromatography from suppliers such as Amersham Biosciences (http://www.amershambiosciences.com, now GE Healthcare) or Bio-Rad (http://www.bio-rad.com).

In a further embodiment of the purification process of the present invention, a membrane with quaternary amino groups is used for anion exchange membrane filtration. The person skilled in the art can obtain product information on suitable matrices and protocols for performing the anion exchange chromatography from the supplier such as Amersham Biosciences (http://www.amershambiosciences.com, now GE Healthcare) or Bio-Rad (http://www.bio-rad.com). In a preferred embodiment, 20 mM sodium acetate pH 5.0 is used for equilibration and washing in the anion exchange chromatography step. Further suitable conditions for anion exchange chromatography can be found in the literature like in the handbook “Ion Exchange Chromatography—Principles and Methods” from Amersham Biosciences, Freiburg, Deutschland (now GE Healthcare), 2002.

For further purification of IFN-β, especially for its use in a pharmaceutical composition, it is advantageous to add further purification and/or concentration steps in particular filtration which are, for example, suitable for the removal of remaining residues from the cell culture such as host cell DNA, endotoxins and other harmful substances. In a preferred embodiment of the present invention, the purification process for IFN-β comprises therefore at least one of the following steps;

(e) an ultrafiltration (UF) step;
(f) a microfiltration (MF) step;
(g) a size exclusion chromatography (SEC) step; and/or
(i) a nanofiltration (NF) step; see also the Examples.

In principle, ultra- and microfiltration serve for the specific purification and concentration of IFN-β while size exclusion chromatography and nanofiltration are especially used for the removal of host cell DNA, endotoxins and remaining process related impurities of the eluates, if present. As explained in the Examples, it is favorable for the purification according to the present invention if ultrafiltration is a tangential flow filtration with a size exclusion of 5 kD-1000 kD, and for microfiltration a 0.2 μm membrane, for the size exclusion chromatography Superdex 200 and/or for the nanofiltration a filter with a pore size of 15-75 nm should be used. Suitable conditions for performing the particular filtration steps as well as the size exclusion chromatography are well known to the person skilled in the art and can be taken from the literature, such as the product monographies from Millipore and Pall Systems. In a preferred embodiment, the claimed purification process for IFN-β comprises the following steps as illustrated in the Examples:

(a) a dye affinity chromatography(AC) step;
(b) a metal chelate affinity chromatography (MAC) step;
(c) a hydrophobic interaction chromatography (HIC) step;
(d) an anion exchange chromatography (AEX) step;
(e) an ultrafiltration (UF) step;
(f) a microfiltration (MF) step;
(g) a size exclusion chromatography (SEC) step;
(f) a microfiltration (MF) step; and
(h) a nanofiltration (NF) step.

The present invention also relates to a pharmaceutical composition, comprising the IFN-β obtained in accordance with the method of the present invention. IFN-β obtained can be stored as lyophilisate or preferably in liquid form. It can be applied subcutaneous or intravenous. Suitable pharmaceutically acceptable carrier for the formulation of recombinantly expressed IFN-β are stabilizers like sugar or sugar alcohols, amino acids as well as tensides like Polysorbate 20 or 80 as well as suitable buffer substances. Examples for formulations are described in international application WO98/28007 and WO99/15193 as well as in European patent application EP 0 529 300, see also products of Avonex® and Rebif® in ROTE LISTE 2005.

Therefore, the present invention also relates to a method for the preparation of a pharmaceutical liquid formulation of human IFN-β suitable for parenteral application comprising a method for the purification of IFN-β as described herein before and in the Examples, and
(i) formulation of the purified IFN-β in a suitable pharmaceutical composition, wherein the IFN-β formulation and optionally further necessary galenic carriers are prepared and brought in a suitable application form and;
(ii) filling or bottling of the formulation into a suitable receptacle or container.

The IFN-β formulation can be stored for example in suitable washed and sterilized glass vials (hydrolytic class 1) with a pharmaceutical acceptable rubber plug. In addition, the pharmaceutical IFN-β formulation can also be filled into antiseptic pre-packaged syringes or in capsules or carpules for self injection devices and used for self injection. The aqueous solution can be freeze-dried—although this is not preferred—by addition of further additional carriers known by the person skilled in the art and is available in liquid form after reconstitution. By addition of suitable preservatives, liquid multiple dosage forms can be produced as well as ophthalmic solutions and drop solutions for oral application. Further carriers needed for the preparation of a suitable dosage form are known to the person skilled in the art; see for example the handbook Remington: The Science and Practice of Pharmacy 20th edition (2000), ISBN 0-683-306472 as well as the patent literature referred to herein above, especially WO 98/28007, and the formulation of the trademark product Avonex©.

In a preferred embodiment the pharmaceutical composition of IFN-β comprises acetate, NaCl or one of the amino acids arginine, lysine and glutamine either alone or in addition to one or more further carriers, wherein the carrier is preferably methionine, mannitol, sorbitol, glycerol or a tenside, wherein the tenside is preferably Polysorbate 20 or 80.

In experiments performed in accordance with the present invention concerning the preparation of a sufficiently stable liquid formulation of IFN-β for storage that the pH value of the formulation preferably ranges between 4.3 and 4.8.

The specific activity of the purified IFN-β according to the present invention is usually at least 1×10⁸ IU/mg, typically at least 2×10⁸ IU/mg, preferably at least 3×10⁸ IU/mg and more.

As illustrated in Example 3, a multitude of possible buffer compositions has been identified as suitable for formulation, providing apparently homogenous IFN-β purified by a dye affinity chromatography (AC), metal chelate affinity chromatography (MAC) and hydrophobic interaction chromatography, which is substantially stable and biologically active at room temperature as well as at −80° C. storage and after subsequent thawing. The stability of IFN-β in a given buffer at a concentration of 200 μg/ml at storage up to two weeks at +4° C. or at −80° C. amounts to at least 95%, preferably at least 97% and possibly nearly 100% of the initial activity. Therefore, the present invention also particularly relates to a pharmaceutical composition comprising biologically active IFN-β.

IFN-β preparations are preferably stable over a storage time of 26 to 27 days at −80° C. and of 47 to 48 days (>6.5 weeks) at +4° C., as confirmed by RP-HPLC measurements, A280 measurement and/or the determination of the A320 value (“turbidity” below 0.010). For both storage conditions, the IFN-β isoform pattern in Western blots after SDS-PAGE and on IEF Western blots is preferably very similar or identical to Avonex© and to the IFN-β preparation after the HIC purification step. By performing an analytical SEC on Superdex 75 HR 10/30 the IFN-β preparations according to the present invention preferably exhibit an apparent molecular mass of 12-16 kDa.

As shown in Example 3, the buffer designated 3a of table 10 is particularly well suited for storage of recombinantly produced glycolysated IFN-β.

Therefore, in a preferred embodiment, the present invention relates to a pharmaceutical composition, comprising IFN-β in 25 mM acetate, 150 mM NaCl and 0.167% (v/v) Polysorbate 20 and which preferably has a pH value of pH 4.8. The liquid pharmaceutical formulations according to the present invention are preferably substantially free of human serum albumin and more preferably—apart from the pharmaceutical agent—free of human or animal polypeptides, in particular of serum proteins.

The stability of the IFN-β formulation can further be positively affected by sparging with an inert gas such as helium or nitrogen. This is particularly true for the present IFN-β formulation in a suitable receptacle or container, wherein the head space of said receptacle or container is preferably also sparged with an inert gas such helium or nitrogen, and preferably wherein the head space is not exceeding 30% of the volume of the receptacle or container.

The present invention also relates to a medicament, comprising purified IFN-β obtained by the method of the present invention and pharmaceutical acceptable carriers as buffer, salts, tensides and stabilizers. As described herein, the liquid formulation of IFN-β is stable over a long time period and can basically be stored in any suitable receptacle or container. Accordingly, the present invention also relates to a receptacle or container comprising a liquid pharmaceutical formulation of human IFN-β suitable for parenteral application and obtainable by method of the present invention for the preparation of the pharmaceutical liquid formulation of IFN-β as described hereinbefore and in particular in the Examples.

The receptacle or container according to the present invention is preferably such that its inner surfaces which are in contact with the pharmaceutical formulation prevent the adsorption of IFN-β. Preferably, at least one surface of the receptacle, or container which is in contact with the liquid formulation, is coated with a material or composed of a material essentially consisting or made of polypropylene (PP), silicone or polytetrafluorethylene or ethylene tetrafluorethylene (ETFE) copolymer.

Typically the receptacle is a container that is conventionally intended for the storage and/or administration of a liquid medicament like a vial, syringe, ampoule, carpule, puncture bottle or infusion container, wherein the liquid formulation of IFN-β according to the present invention is particularly advantageous for the use in pre-filled syringes or ampoules. In a preferred embodiment, the liquid formulation is present for example in a syringe or an ampoule at a concentration of IFN-β of 10-500 μg/ml, preferably 50-250 μg/ml and/or an activity of 5-50 million I.E./ml.

The pharmaceutical compositions of the present invention obtained as well as the receptacles and containers containing these compositions can be used for the treatment of tumors, virus diseases, immunopathies or inflammations including rheumatic diseases, allergies, psoriasis, Crohn's disease and degenerative diseases of the nervous system, in particular multiple sclerosis. The required quantity of recombinant IFN-β in a medicament for the desired therapeutic effect depends on the respective administration and treated subject as well as the respective disease. A suitable dosage of the active integrient for administration on a human is ranging between 0.1×10⁶ and 100×10⁶ I.E. The most preferred dosage, in particular in case of a local therapy is 6×10⁶ I.E., for systemic therapy approximately 1×10⁶ to 30×10⁶ I.E. per day, if appropriate several times per day.

Pharmaceutical compositions according to the present invention and the receptacles and containers containing them, are preferably designed for ophthalmological, subcutan, intracutan, intramuscular, intravenious, intrathecal, intraarticular, intratumoral/peritumoral, intralesional/perilesional or topic application.

As advantageously no further carriers are admixed or further preparatory measures taken, like filtration, mixing, etc. prior to the administration of the liquid formulation of IFN-β according to the present invention, the IFN-β liquid medicament according to the present invention can be used for immediate administration, for example in a kit.

In one embodiment that is particularly advantageous for physicians, pharmacists and especially for patients, the present invention therefore also relates to a kit for the administration of IFN-β by infusion or injection, comprising one or more of the above described receptacles, preferably along with instructions for storage and/or administration. Usually IFN-β administration at a dosage of 1×10⁶ to 10×10⁶ I.E. will be provided, wherein also lower or higher dosages may be indicated, however, depending on the medical indication and stage of disease. Preferably, several receptacles are provided in the kit according to the present invention, for example for weekly intramuscular administration for one month 4 pre-filled syringes with needles or 4 puncture bottles along with pre-packed syringes and needles as well as, if appropriate, a solvent in case lyophilisate is used which is basically possible, but however not preferred. By contrast, Rebif© is intravenously administrated three times a week.

For reasons of safe handling, the kit according to the present invention advantageously has safety compartments for syringes, injections and/or infusions needles, respectively. Here, discharge aids for the needles and prepared or pre-fitted sealing caps are also to be considered.

As described in the Examples, the liquid IFN-β formulations according to the present invention are stable over a long time period, in particular at about 2-8° C., preferably over a period of at least 4 weeks. Therefore, the liquid formulations, receptacles and kits according to the present invention can advantageously be stored in a conventional refrigerator.

These and further embodiments resulting from the present invention are encompassed by the claims.

The disclosure content of the prior art documents cited above and in the following is herewith incorporated in the present application by reference, in particular with respect to the recombinant production of IFN-β, buffers, syringes and kits. These and further embodiments are disclosed and apparent to the person skilled in the art and are encompassed by the description and the Examples of the present invention. Further literature on one of the above-mentioned carriers as well as on electronic means that can be used in accordance with the present invention can be taken from the prior art, for example from public libraries using, e.g. electronic means. In addition, further public databases are readily available via the interne, like for example “Pubmed” (http://www.pubmed.gov).

Techniques for performing the present invention are known for the person skilled in the art and can be taken from relevant literature, see for example Molecular Cloning. A Laboratory Manual, 2nd Ed., ed. by Sambrook, Fritsch and Maniatis (Cold Spring Harbor Laboratory Press: 1989); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

In the following, the present invention is further illustrated by way of a preferred embodiment which, is, however, not intended to limit the scope of the invention in any way.

### EXAMPLE 1

### Provision of Starting Material for the Purification of IFN-β

IFN-β as a starting point typically is human recombinant IFN-β1a expressed by recombinant Chinese Hamster Ovary (CHO) cell line. The protein contains arginine N-linked glycanes, exhibiting an isoform profile as described in the relevant draft Ph.Eur. monograph (PHARMEUROPA) Vol. 15, Nr. 4, October 2003. The specific activity is demonstrated in the CPE assay (CPE, cytopathic effect) by use of A549 cells and encephalomyocarditis virus EMC as infections agent (PHARMEUROPA, Vol. 15, Nr. 4, October 2003). The specific activity of purified bulk IFN-β is approximately 3.2×10⁸ IU×mg⁻¹.

A recombinant CHO cell line is generated, which expresses human IFN-β1a, and is adapted to suspension and serum-free culture conditions. As expression vector, a vector can be used which contains the (natural) human IFN-β gene sequence, a translation start sequence according to Kozak (Kozak sequence) and as a regulatory element for expression the SV40 promoter with a SV40 polyA terminator sequence. The selection and amplification of the expression vectors take place by a murine dhfr gene sequence under the control of an Adeno major late promotor and the SV40 polyA sequence. The parental cell line is preferably a dhfr-deficient Chinese Hamster Ovary (CHO) cell line obtainable for example from ATCC or DSMZ. The generation of the producer cell line is made in accordance with methods known in the art. The dhfr deficient CHO cells are transfected with the expression vector. After selection, subcloning utilizing cloning cylinders and subsequent amplification using methotrexate, the resulting cell line is adapted to serum free culture condition and is tested as suspension culture. A second round of subcloning can be performed utilizing the limited dilution technique.

The cell culture process consists preferably of seed train expansion of transfected CHO cells in Erlenmeyer flasks followed by commercial scale production in a 10 liter bioreactor. Growth and IFN-β expression in a perfusion bioreactor with 1 liter in pilot scale and 10 liter in commercial scale of working volume over a period of 4 weeks then takes place. When cell density preferably reaches 1.2 (+0.2)×10⁶ cells/ml, cell culture media is continuously harvested by perfusion through acoustic cell retention and continuously collected, stored in 50 to 200 liter Stedim bags at 5±3° C. The product is weekly captured by Blue Sepharose affinity chromatography and eluates are frozen at <−70° C. until they are subjected to subsequent purification procedure. Four above described Blue Sepharose eluates derived from a complete perfusion fermentation run are thawed, pooled and subjected to the next purification step.

### EXAMPLE 2

### Purification of IFN-β from Cell Culture Supernatant

The purification process comprises several steps designed to yield a product with high biological activity and biochemical characteristics, and which at the level of product—and product—related substances and impurities are in full compliance with the current regulatory, scientific and compendia standards. The scope for this development phase includes an extensive optimization of each purification step and to generate the representative material for a pilot PK/PD-study with Cynomolgus monkeys. Due to limited development resources, the purification (excluding capture step) of this material is performed in a scaled down laboratory process.

### a) Affinity Chromatography: Streamline Blue Sepharose

For capturing phenyl and Blue Sepharose Fast Flow is tested. Best results are obtained by using Blue Sepharose resin and a stepwise washing and elution of the column with 10%, 20% and 50% ethyleneglycol, respectively. The harvest (H) derived from 7 day collection (i.e. H1 to H4) is first captured by affinity chromatography, using Blue Sepharose FF resin (GE Healthcare), are specified in table 1. In order to load the crude harvest directly onto a resin without any additional separation of residual cells from the harvest, the Streamline® technology is investigated using Streamline® Blue Sepharose. Modification and adaptation of chromatographic parameters (e.g. flow rate, expansion factor, contact time, conductivity of the load sample and amount) let to the final method described in the following table 1. The step yield is usually above 60%.

TABLE 1
Capture-Chromatography: Streamline Blue Sepharose

| Parameter | Criterion | |
|---|---|---|
| Resin | Streamline © Blue Sepharose | |
| Column diameter | 10 cm | |
| Bed height | 20.4 ± 1.5 cm | |
| Theoretical plates | n.a. | |
| Assymmetry factor | n.a. | |
| Column temperture | 22 ± 3° C. | |
| Flow rate | 120 L/hr—all stages (Column bypass 120 L/h) | |
| Loading buffer | 20 mM NaH₂PO₄/Na₂HPO₄, pH 7.2, 1M NaCl | |
| Washing buffer | a) | 20 mM NaH₂PO₄/Na₂HPO₄, 1M NaCl, |
| | | pH 7.2 |
| | b) | 20 mM NaH₂PO₄/Na₂HPO₄, 1M NaCl, |
| | | pH 7.2, 10% ethylene glycol |
| | c) | 20 mM NaH₂PO₄/Na₂HPO₄, 1M NaCl, |
| | | pH 7.2, 20% ethylene glycol |

| | |
|---|---|
| Eluation buffer | 20 mM NaH₂PO₄/Na₂HPO₄, pH 7.2, 1M NaCl, |
| | 50% ethylene glycol |
| Eluation volume | 12.8 L |
| Collection | Step elution, total peak collection |

For equilibration a washing buffer is used a): 20 mM NaH₂PO₄/Na₂HPO₄, 1M NaCl, pH 7.2. Column regeneration is performed with regeneration buffer 1: 50 mM Tris/HCl, 1 M NaCl, pH 7.6; regeneration buffer 2: 10 mM Tris-HCl, 800 mM NaCl, 50 mM EDTA, 10% Isopropanol, pH 7.6 and regeneration buffer 3: 50 mM Tris/HCl, 3 M NaCl, pH 7.6 together with SIP: 70% Ethanol >12 h and 2 h/CIP: 0.5 M NaOH. Subsequently the column is stored in storage buffer: 0.01 M NaOH preferably at 2-8° C.

TABLE 2
General time flow of purification after capture-step

| Day −x to 0 | Storage of Blue Sepharose Capture eluates at | | <−70° C. |
|---|---|---|---|
| Day 0 | 1) | Thawing of Blue Sepharose Capture | 5 ± 3° C. |
| | | eluates | |
| | 2) | Sanitisation of Chelating Sepharose | |
| Day 1 | 1) | Chelating-Sepharose-Chromatography | 5 ± 3° C. |
| | 2) | Butyl Sepharose sanitisation | |
| Day 2 | 1) | Chelating Sepharose eluate | 5 ± 3° C. |
| | | conditioning (addition of EDTA) | |
| | 2) | Butyl Sepharose-Chromatography | |
| | 3) | Sanitisation and equilibration of | |
| | | Mustang Q membrane absorber | |
| | 4) | Mustang Q membrane filtration | |
| | | (percolation mode) | |
| | 5) | Sanitisation of ultrafiltration device | |
| | 6) | Sanitisation of SEC | |
| Day 3 | 1) | Ultrafiltration | RT |
| | 2) | Mikrofiltration | |
| | 3) | SEC | |
| Day 4 | 1) | Nanofiltration | |
| | 2) | Adjustment of concentration to 0.200 | |
| | | μg/ml | |
| | 3) | Sterile filtration | |
| | 4) | Filling | |
| | 5) | Freezing | RT |

### b) Affinity Chromatography: Chelat-Sepharose FF

After comprehensive analysis for establishing metal chelate affinity chromatography process by use of zinc- and copper-charged chelating Sepharose, including optimization of salt content and elution steps, binding to zinc Sepharose is very selective, and after elution with a pH 5.0 buffer, IFN-β is 96% homogeneous, and resulted in a step yield of approximately 75%. Therefore, the four Blue Sepharose eluates (out of a complete 10 liter perfusion fermentation ground) are pooled and applied onto a Zn²⁺ chelate Sepharose FF column (GE Healthcare). Further optimization of the loaded sample amount and replacement of the phosphate buffer system by an acetate based buffer system result in the method described below with step yield of up to above 95%. In addition, host cell proteins essentially are removed.

TABLE 3
Affinity chromatography: Chelate Sepharose FF

| Parameter | Criterion |
|---|---|
| Resin | Chelate Sepharose FF |
| Column diameter | 14 cm |
| Bed height | 24 ± 1.5 cm |
| Theoretical plates | >3500 N/m |
| Assymmetry factor | n.a. |
| Column temperature | 22 ± 3° C. |
| Flow rate | 17.58 L/hr—all stages |
| Loading buffer | 20 mM Na-acetate, pH 5.0, 1M NaCl |
| Washing buffer | a) 20 mM NaH₂PO₄/Na₂HPO₄, 1M NaCl, pH 7.2 |
| | b) 20 mM NaH₂PO₄/Na₂HPO₄, 1M NaCl, pH 6.5 |
| Elution buffer | 20 mM Na-acetate, pH 5.0, 1M NaCl |
| Elution volume | 12.8 L |
| Collection | Step elution, total peak collection |

Prior to loading, the column is equilibrated with water, Zn(Cl₂), water and loading buffer. For cleavage of zinc the column is incubated with 50 mM EDTA, 1M NaCl followed by 0.5 M NaOH. The column is stored in 20 mM NaOH storage buffer.

### c) Hydrophobic Interaction Chromatography: Butyl-Sepharose FF

The feasibility of using hydrophobic interaction chromatography on Butyl Sepharose FF is demonstrated yielding in approximately 60% after optimization of buffer system and temperature. Therefore, hydrophobic interaction chromatography, using butyl Sepharose resin (GE Healthcare), is performed.

After incorporation of a special wash step to remove contaminants and performing the elution in upflow mode, the method resulted in step yield of up to 80%. In addition remaining CHO/HCP contaminates could be removed.

TABLE 4
Hydrophobic Interaction chromatography: butyl Sepharose FF

| Parameter | Citerion | |
|---|---|---|
| Resin | butyl Sepharose FF | |
| Column | 18 cm | |
| diameter | | |
| Bed height | 10 ± 0.5 cm | |
| Theoretical | >3.500 N/m | |
| plates | | |
| Assynnetry | n.a. | |
| factor | | |
| Column | 5 ± 3° C. | |
| temperature | | |
| Flow rate | a) | 22.8 L/h—all stages exept elution flow rate |
| | b) | 2.28 L/h elution flow rate |
| Loading buffer | 20 mM Na-acetate, pH 5.0, 1M NaCl, 1 mM EDTA | |
| Washing buffer | a) | wash I after sample loading: 20 mM Na-acetate, |
| | | pH 5.0, 1M NaCl, 1 mM EDTA |
| | b) | wash II: 20 mM NaH₂PO₄/Na₂HPO₄, pH 8.0, |
| | | 1M NaCl, 1 mM EDTA |
| | c) | wash III: 20 mM Na-Acetat, pH 5.0, 0.75M |
| | | NaCl |

| | |
|---|---|
| Elution buffer | 20 mM Na-acetate, pH 5.0, Upflow-Modus |
| Elution volume | 7.6 liter |
| Collection | Complete step gradient is collected (3 column |
| | volumes) |

Conductivity of the eluate is determined and adjusted to 2.5±0.5 mS/cm using 20 mM Na-acetate, pH 5.0 (approximately dilution factor 2). The pH is adjusted to pH 5.0±0.1. The column is equilibrated with washing buffer a) and after elution of the samples treated with SIP/CIP: 0.1M NaOH and is stored in storage buffer: 20 mM NaOH.

### d) Anion Exchange Membrane Filtration

In order to remove host cell DNA and potential virus contamination, an AEX membrane filtration is introduced directly after the HIC step. During a pre-validation study of a viral infectivity Mustang Q is identified to be a potent method for viral clearance in precolation mode. Anion exchange chromatography of butyl Sepharose eluate is performed and specified in table 5 using a Mustang Q filter cartridge (Pall system), yielding in approximately 97%.

TABLE 5
Anion exchange membrane filtration (Percolation modus)

| Parameter | Criterion |
|---|---|
| Filter | Mustang Q |
| Filter type | Dead-End |
| Pore size | 0.8 μm |
| Filter area | 260 cm³ |
| Flow rate | 0.3-0.5 L/min |
| Temperature | 22 ± 3° C. |
| Membrane sanitizatiation | 1M NaOH, 3 liter |
| Loading buffer | 20 mM Na-acetate, pH 5.0 (HP-Flow) |
| Washing buffer | 20 mM Na-acetate, pH 5.0 |
| Regeneration/Cleaning | 20 mM Na-acetate, pH 5.0, 1M NaCl |
| Filtrate volume | 15-16 liter |

Loading buffer is used for equilibration.

### e) Ultrafiltration

For concentration of IFN-β in Mustang Q filtrate before Size Exclusion Chromatography (SEC) an ultrafiltration step using a polyethersulfone (PES) membrane is established in tangential flow mode. This method resulted in a step yield of about 80 to 100% as explained in table 6.

TABLE 6
Ultrafiltration: Concentration of Mustang Q Eluate

| Parameter | Criterion |
|---|---|
| Apparatus | ProFlux M12 |
| Filter | Pellicon2 Biomax screen A |
| | Millipore Cat: P2B010A05 |
| Cut-off | 2 × 0.5 m² |
| Filter area | 0.3 ± 0.2 bar |
| Feed pressure | 0.1 ± 0.2 bar |
| Retentate pressure | 2 × 0.5 m² |
| Final concentration [μg/ml] | 3261-4433 |

Finally, the system is washed with 20 mM NaAc, pH 5.0.

### f) Microfiltration

In order to remove precipitates and eliminate potential bio burden, three microfiltrations are introduced into the purification process, before SEC eluate is filtrated through a 0.22 μm PES-membrane (Pall Systems). Here again, for final washing of the system 20 mM NaAc, pH 5.0 is used.

### g) Size Exclusion Chromatography: Superdex 200

Size exclusion chromatography on Superdex 75, Superdex 200 and Sephacryl S 100 is investigated as a polishing step. Contaminating proteins are quantitatively separated from IFN-β, yielding up to 85%. With the respect to the demand of a small elution volume, Superdex 200 proved to be the most suitable resin. As a consequence, the CEX (SP-Sepharose) is replaced by a SEC, since yields of approximately 25% are only obtained using the CEX-technique.

Therefore, size exclusion chromatography, using Superdex 200 resin (GE Healthcare), is performed and specified in table 7. This step reduces CHO/HCP contaminations. Since the total amount of IFN-β needed for pre-clinical purpose is very small (approximately 200 mg), this process step is scaled down by the factor 4.6. This procedure also reduces project costs. Based on the developments during laboratory scale, the loaded sample volume is defined to be 2.5% of the column volume. Yields between 86 and 96% are obtained.

TABLE 7
Size exclusion chromatography: Superdex
200 (down scaled by factor 4.6)

| Parameter | Criterion |
|---|---|
| Resin | Superdex 200 |
| Column diameter | 140 cm |
| Bed height | 60 ± 3 cm |
| Theoretical plates | >10.000 N/m |
| Assymmetry factor | 0.6 < As < 1.8 |
| Column temperature | 22 ± 3° C. |
| Flow rate | 3.492 L/h—all stages |
| Running buffer | 25 mM NaAc, pH 4.8, 0.15M NaCl 0.167% (v/v) |
| | Tween 20 |
| Loading volume | maximum 2.5% of column volume |
| Elution volume | 1.1-1.4 liter |
| Collection | peak start-peak end |

Loading buffer used for equilibration is sparged with nitrogen in order to remove oxygen prior to equilibration, and after subsequent elution of the sample the column is treated with 1M NaOH and is stored in storage buffer: 20 mM NaOH.

### f) Pre-Filtration/Nanofiltration

The purified material is filtered prior to nanofiltration according to table 8. Mini Kleenpak (Pall Systems) is used during filtration in order to protect the nanofiltration membrane for micro-precipitates.

TABLE 8
Pre-filtration (Microfiltration) of purified material

| Parameter | Criterion |
|---|---|
| Filter | Mini Kleenpak PALL cat: KA02 EKVP2S |
| Filter type | PES (polyethersulfone) |
| Pore size | 0.2 μm |
| Filter area | 0.022 m² |
| Flow rate | 250-300 ml/min |

After the pre-filtration the purified material is filtered through a Planova N20 filter device (table 9). For virus filtration the hollow fiber system of Planova 20N (Asahi Kasei) is appropriate regarding the yield to IFN-β and the compatibility with a use drug substance storage buffer. This method yields in 90 to 100%. The purity was >99% (determined by RP-HPLC).

TABLE 9
Nanofiltration of purified material

| Parameter | Criterion |
|---|---|
| Filter | Planova N20 |
| Filter type | Hollow fibre, dead end |
| Pore size | 20 nm nominal |
| Filter area | 1200 cm² |
| Flow rate | 0.45 ± 0.05 |

The nanofilter is washed with up to 300 mL 20 mM NaAc, pH 5.0, 150 mM NaCl, 0.167% Tween 20 (v/v). Thereafter, the purified drug substance is filled into containers for freezing and storage (TPP Cryotubes).

The total yield of IFN-β in relation to the starting activity is 25%.

### EXAMPLE 3

### Identification of a Suitable Storage Buffer System for IFN-β Drug Substance

The aim of this study is the identification of a buffers system and of storage conditions suitable for IFN-β drug substance.

IFN-β Blue Sepharose eluates are purified by zinc Sepharose chromatography and butyl

Sepharose chromatography and are concentrated to approximately 1.9 mg/ml using Vivacell 70 centrifugal filter device. This preparation is the starting material needed for the formulation studies.

The buffer exchange is performed using Size Exclusion Chromatography (NAP10, GE Healthcare). A total of 20 different buffers are investigated (see table 10), partly sparged with nitrogen as inert gas. Furthermore, the influence of the head space and closure system on storage stability and precipitation is analyzed.

TABLE 10
Conditions for the identification of a buffer
system for stable storage of IFN-β

| | | | oxidizing/ |
|---|---|---|---|
| | | c_{IFN-β} | non-oxidizing |
| Nr. | Buffer composition | [μg/ml] | conditions* |
| 1 | 25 mM acetate, 150 mM NaCl, pH 4.8 | 200 | oxidizing |
| 2 | 25 mM acetate, 150 mM NaCl, pH 4.8 | 200 | non-oxidizing |
| 3a | 25 mM acetate, 150 mM NaCl, 0.167% | 200 | non-oxidizing |
| | Tween 20, pH 4.8 | | |
| 3b | 25 mM acetate, 150 mM arginine, | 200 | non-oxidizing |
| | 0.005% Tween 20, pH 4.8 | | |
| 3c | 25 mM acetate, 150 mM Lysine, | 200 | non-oxidizing |
| | 0.005% Tween 20, pH 4.8 | | |
| 4a | 25 mM acetate, 150 mM NaCl, 0.005% | 200 | non-oxidizing |
| | Tween 20, 300 mM mannitol, pH 4.3 | | |
| 4b | 25 mM acetate, 10% (v/v) ethanole, pH | 200 | non-oxidizing |
| | 4.3 | | |
| 5 | 25 mM acetate, 100 mM CaCl₂, pH 4.3 | 200 | non-oxidizing |
| 6 | 25 mM acetate, 150 mM NaCl, 200 mM | 200 | non-oxidizing |
| | CHES, pH 4.3 | | |
| 7 | 25 mM acetate, 150 mM NaCl, 150 mM | 200 | non-oxidizing |
| | betain, pH 4.3 | | |
| 8 | 25 mM acetate, 150 mM NaCl, 150 mM | 200 | non-oxidizing |
| | GlutaMAX-II, pH 4.3 | | |
| 9aI | 25 mM acetate, 150 mM NaCl, 25% | 200 | non-oxidizing |
| | (v/v) glycerine, pH 3.0 | | |
| 9aII | 25 mM acetate, 150 mM NaCl, 25% | 200 | non-oxidizing/ |
| | (v/v) glycerine, pH 4.8 | | semi-sterile |
| 9bI | 25 mM acetate, 150 mM NaCl, 25% | 200 | non-oxidizing |
| | (v/v) PEG300, pH 3.0 | | |
| 9bII | 25 mM acetate, 150 mM NaCl, 25% | 200 | non-oxidizing/ |
| | (v/v) PEG300, pH 4.8 | | semi-sterile |
| 10 | 25 mM acetate, 150 mM NaCl, 300 mM | 200 | non-oxidizing |
| | glucose, pH 4.3 | | |
| 11aI | 70 mM Citrate, 50 mM NaH₂PO₄, | 60 | non-oxidizing |
| | 2 mM methionine, 200 mM glycerine, | | |
| | pH 5.0 | | |
| 11aII | 70 mM Citrate, 50 mM NaH₂PO₄, | 60 | non-oxidizing |
| | 2 mM methionine, 200 mM glycerine, | | |
| | pH 6.5 | | |
| 11b | 50 mM Imidazol, 200 mM NaCl, | 350 | non-oxidizing |
| | pH 7.5 | | |
| 11c | 50 mM NaH₂PO₄, 100 mM NaCl, | 150 | non-oxidizing |
| | pH 7.5 | | |
| 12 | 50 mM glycine, 2 mM lanthanum (III) | 200 | non-oxidizing |
| | chloride, pH 3.0 | | |

*Non-oxidizing conditions: the buffers are 0.2 μm filtered using vacuum filtration and sparged with N₂ for 10 min before use; after filing of the formulated IFN-β into the PP cryovials the head space (maximally 30% of the cryovial volume) is filled with 0.2 μm filtered N₂. Oxidizing conditions: Buffers not 0.2 μm are filtered using vacuum filtration, but are sparged with air for 10 min before use; after filling of the formulated IFN-β into the PP cryovials the head space are not filled with N₂. Non-oxidizing/semi-sterile conditions: The buffers are first sparged with N₂ for 30 min, then sterile filtered through a 0.2 μm cellulose acetate membrane (Schleicher & Schuell; FP 30/02 CA-S) under a lamina flow; the following work are performed under a lamina flow; the final protein solution are not sterile filtered because a significant loss of IFN-β is expected; after filling of the formulated IFN-β into the PP cryovials the head space (maximally 30% of the cryovial volume) is filled with 0.2 μm filtered N₂.

IFN-β protein stability measured by the means of RP-HPLC measurement, showing good stability for all buffer eluates after storage for 12 to 14 days at +4° C. (97-103% recovery) with the exception of buffer 11 aI which exhibit a lower IFN-β concentration (approximately 60 μg/ml) and small amounts of stabilizers. Good stability is also shown for all buffer eluates after storage for 10 to 14 days at −80° C. (98-102% recovery) with the exception of 6 buffer eluates, which exhibit a low IFN-β concentration (approximately 60 μg/ml) or only small amounts of stabilizers like 10% (v/v) ethanole, 100 mM CaCl₂, 200 mM CHES or 50 mM imidazol, respectively.

The A320 values (“turbidity”) is especially low (0.000-0.006) for buffer eluates 3a (25 mM acetate, 150 mM NaCl, 0.167% Tween 20, pH 4.8), 9aI (25 mM acetate, 150 mM NaCl, 25% (v/v) glycerol, pH 3.0) and 9bI (25 mM acetate, 150 mM NaCl, 25% (v/v) PEG300, pH 3.0) before the storage and after storage at both +4° C. and −80° C., and for two more buffer eluates containing low IFN-β concentration (approximately 60 μg/ml) before the storage and after storage at +4° C.

Further analysis (RP-HPLC, % T580, A280, A320, Western blot after SDS-PAGE, IEF Western blotting, analytical SEC on Superdex 75 HR 10/30 and Peptide mapping) are performed for buffer 3a, 9aI and 9bI eluates after the total storage time of 26 to 27 days for the −80° C. samples and 47 to 48 days (>6.5 weeks) for the +4° C. samples.

RP-HPLC measurement (100-102% recovery) and the A280 measurement (99-107% recovery) show a very good stability (IFN-β protein recovery) for all three buffer eluates as well as for both storage conditions. The A320 value (“turbidity”) was very low (between 0.000 and 0.008) for all 3 buffer eluates as well as the 2 different storage conditions.

Western blotting after SDS-PAGE and IEF Western blots are performed for all buffer eluates after both storage conditions and show a very similar isoform pattern, which is also very similar to the Avonex and to the concentrated HIC eluate control (control “before buffer exchange”).

IFN-β elutes in all 3 buffer eluates in both different storage conditions as well as in the HIC eluate control (control “before buffer exchange”) with an apparent molecular mass of around 12-16 kDa in an analytic SEC on Superdex 75 HR 10/30.

In summary, the most suitable buffer for the drug substance with respect to a storage of IFN-β at −80° C. (a demand of the preliminary monograph for IFN-β) regarding the yield after thawing and the absence of precipitation is used for pilot and commercial scale (25 mM NaAc, pH 4.8, 150 mM NaCl, 0.167% Tween 20).

1. A method of purifying human interferon-β (IFN-β), comprising at least one affinity chromatography (AC) step and at least one hydrophobic interaction chromatography (HIC) step, wherein the affinity chromatography step(s) and the hydrophobic interaction chromatography step(s) are applied immediately succeeding one another in either order.

2. The method according to claim 1, comprising two affinity chromatography steps performed before the hydrophobic interaction chromatography step.

3. The method according to claim 1, further comprising an anion exchange chromatography (AEX) step.

4. The method according to claim 3, wherein the anion exchange chromatography step is performed after the hydrophobic interaction chromatography step(s).

5. The method according to claim 1, further comprising at least one:
(a) color ligand affinity chromatography (AC) step;
(b) metal chelate affinity chromatography (MAC) step;
and/or
(c) anion exchange membrane filtration step.

6. The method according to claim 5, wherein for the color ligand affinity chromatography (AC) step, Cibacron Blue is used; for the metal chelate affinity chromatography (MAC) step, Zn-chelate Sepharose (IMAC) is used; and for the hydrophobic interaction chromatography (HIC) step, butyl groups are used as ligands; and/or for the anion exchange membrane filtration step, a membrane with quaternary amino groups is used.

7. The method according to claim 1, wherein no HPLC step is performed.

8. The method according to claim 1, wherein no cation exchange chromatography step is performed.

9. The method according to claim 1, wherein no hydroxyapatite chromatography step is performed.

10. The method according to claim 1 further comprising at least one of the following steps:
(a) an ultrafiltration (UF) step;
(b) a microfiltration (MF) step;
(c) a size exclusion chromatography (SEC) step; and/or
(d) a nanofiltration (NF) step.

11. The method according to claim 10, wherein for the ultrafiltration step, a tangential flow filtration comprising an exclusion size of 5 kD-1000 kD; for the microfiltration step, a 0.2 μm membrane; for the size exclusion chromatography step, Superdex 200; and/or for the nanofiltration step, a filter with a pore size of 15-75 nm is or are used.

12. The method according to claim 1 further comprising the following steps:
(a) a color ligand affinity chromatography (AC) step;
(b) a metal chelate affinity chromatography (MAC) step;
(c) an anion exchange chromatography (AEX) step;
(d) an ultrafiltration (UF) step;
(e) a microfiltration (MF) step;
(f) a size exclusion chromatography (SEC) step;
(g) a microfiltration (MF) step; and
(h) a nanofiltration (NF) step.

13. The method according to claim 1, wherein IFN-β is produced by a eukaryotic host cell.

14. The method according to claim 13, wherein said eukaryotic host cell is a Chinese Hamster Ovary (CHO) cell.

15. A method for the preparation of a liquid pharmaceutical formulation of human IFN-β suitable for parenteral application comprising a method of purification of IFN-β according to any of the preceding claims, and
(i) formulation of purified IFN-β in a suitable pharmaceutical composition; and
(ii) bottling of the formulation in a suitable receptacle.

16. The method according to claim 15, wherein the pharmaceutical composition comprises acetate, NaCl or one of the amino acids arginine, lysine and glutamine alone or with one or more pharmaceutically acceptable carrier(s).

17. The method according to claim 16, wherein the carrier is methionine, mannitol, sorbitol, glycerol or a tenside.

18. The method according to claim 17, wherein the tenside is Polysorbate 20 or 80.

19. The method according to claim 15, wherein the pH value of the formulation is between 4.3 and 4.8.

20. The method according to claim 15, wherein the pharmaceutical composition comprises 25 mM acetate, 150 mM NaCl and 0.167% (v/v) Polysorbate 20.

21. The method according to claim 15, wherein the formulation is devolatilized with an inert gas such as helium or nitrogen.

22. The method according to claim 15, wherein the head space is sparged with an inert gas such as helium or nitrogen.

23. The method according to claim 22, wherein the headspace does not exceed 30% of the volume of the container.

24. A medicament comprising IFN-β purified, in accordance with the method of claim 1 and a pharmaceutically acceptable carrier.

25. A receptacle comprising a liquid pharmaceutical formulation of human IFN-β suitable for parenteral administration obtainable in accordance with a method of claim 15.

26-29. (canceled)

30. A kit for administration of IFN-β via injection or infusion, comprising a receptacle according to claim 25 and instructions for storage and/or administration.

31-33. (canceled)
